(19) United States
(12) Patent Application Publication
(10) Pub. No.: US 2026/0186007 A1
(43) Pub. Date: Jul. 2, 2026

(54) DISEASE-SPECIFIC METABOLITE FOR DIAGNOSING AND DETERMINING SEVERITY OF PRE-ECLAMPSIA, AND USE THEREOF

(71) Applicant: SUNGKWANG MEDICAL FOUNDATION, Seoul (KR)

(72) Inventors: Ji Hyae LIM, Seongnam-si Gyeonggi-do (KR); Hyun Mee RYU, Seongnam-si Gyeonggi-do (KR)

(73) Assignee: SUNGKWANG MEDICAL FOUNDATION, Seoul (KR)

(21) Appl. No.: 19/546,736

(22) Filed: Feb. 23, 2026

Related U.S. Application Data
(63) Continuation of application No. PCT/KR2024/012511, filed on Aug. 22, 2024.

(30) Foreign Application Priority Data
Aug. 23, 2023 (KR) 10-2023-0110546

(51) Int. Cl. G01N 33/92

(52) U.S. Cl. CPC G01N 33/92; G01N 2570/00; G01N 2800/368

(57) ABSTRACT

The present disclosure relates to a disease-specific lipidome for the diagnosis and severity determination of pre-eclampsia. A composition for diagnosing pre-eclampsia and a composition for determining the severity of pre-eclampsia, according to an aspect, can diagnose pre-eclampsia and significantly determine the severity of the disease, by measuring and comparing the level of a lipidome that changes in patients.

### CROSS-REFERENCE TO RELATED APPLICATIONS

This application is a continuation of International Application No. PCT/KR2024/012511 filed on Aug. 22, 2024, which claims priority to Korean Patent Application No. 10-2023-0110546 filed on Aug. 23, 2023, the entire contents of which are herein incorporated by reference.

### TECHNICAL FIELD

The present disclosure relates to a disease-specific lipidome for the diagnosis and severity determination of pre-eclampsia, and use thereof.

### BACKGROUND ART

Lipidomics is an academic field that focuses on qualitative and quantitative analysis of the overall phenomena and changes that occur in the metabolic processes of various lipidomes occurring in the body to determine their biological and biochemical significance, with the aim of conducting research to find biomarkers related to various diseases. Particularly, with the remarkable recent advancements in qualitative and quantitative analysis methods, it has become possible to qualify and quantify lipids at a sophisticated level and analyze the lipids by using statistical and bioinformatic methods.

Pre-eclampsia (PE) is a hypertensive disorder (>140/90 mmHg) that develops after 20 weeks of gestation, accompanied by proteinuria (>300 mg/day) due to various causes such as inflammation, hypoxia, and oxidative stress, and is a major cause of maternal death occurring in 7-12% of pregnant women and also accounts for a large proportion of deaths and morbidities in fetuses and newborns. Progression to severe pre-eclampsia may lead to pulmonary edema, cerebral hemorrhage, liver and kidney failure, coagulation disorders, and even maternal death. In addition, pre-eclampsia causes disruption of blood flow to the placenta and fetus, resulting in fetal growth restriction and, in severe cases, fetal death. However, the precise pathogenesis and treatment method of pre-eclampsia remain unidentified to date, and the only treatment method is to terminate the pregnancy by delivery. However, the development of pre-eclampsia can increase the risk of fetal mortality due to premature birth, and thus the best method is to diagnose the disease early and delay delivery as much as possible.

In pregnant women after 20 weeks of pregnancy (more frequently occurring after 32 weeks), it is possible to diagnose pre-eclampsia by using various indicators such as hypertension, proteinuria, and edema, but an early pre-eclampsia diagnosis method for reducing premature birth caused by pre-eclampsia has not yet been clearly established. Currently, tests using plasma markers have been developed as the early pre-eclampsia diagnosis method, but the PerkinElmer DELFIA® Xpress PlGF kit has a false positive rate of 5% and a detection rate of 44%, and the Alere Triage® PlGF test has insufficient clinical diagnostic efficacy. Thus, the current methods still have low accuracy and lack clinical efficacy. In addition, therapeutic approaches to pre-eclampsia vary depending on the severity thereof, and thus there is a need for a test method of diagnosing pre-eclampsia according to the severity thereof. However, there is no prior art developed to date.

Therefore, the inventors of the present disclosure have discovered the world's first pre-eclampsia-specific lipidome that shows a significant change in patients, and have developed a simple and effective method of diagnosing pre-eclampsia and significantly determining the severity of the disease.

### DISCLOSURE OF INVENTION

### Technical Problem

An aspect is to provide a composition for diagnosing pre-eclampsia, including an agent for measuring the level of a lipidome.

Another aspect is to provide use of an agent for measuring the level of a lipidome for diagnosing pre-eclampsia.

Another aspect is to provide use of an agent for measuring the level of a lipidome for preparing a composition for the diagnosis of pre-eclampsia.

Another aspect is to provide a composition for determining the severity of pre-eclampsia, including an agent for measuring the level of a lipidome.

Another aspect is to provide use of an agent for measuring the level of a lipidome for determining the severity of pre-eclampsia.

Another aspect is to provide use of an agent for preparing a composition for determining the severity of pre-eclampsia, the agent being for measuring the level of a lipidome.

Another aspect is to provide a kit for diagnosing pre-eclampsia, including the composition.

Another aspect is to provide a kit for determining the severity of pre-eclampsia, including the composition.

Another aspect is to provide a method of diagnosing pre-eclampsia, including measuring the level of a lipidome in a sample isolated from a subject, and comparing the measured level of the lipidome with that of a normal control.

Another aspect is to provide a method of providing information for the diagnosis of pre-eclampsia, the method including measuring the level of a lipidome in a sample isolated from a subject, and comparing the measured level of the lipidome with that of a normal control.

Another aspect is to provide a method of determining the severity of pre-eclampsia, including measuring the level of a lipidome in a sample isolated from a subject, and comparing the measured level of the lipidome with that of a normal control.

Another aspect is to provide a method of providing information for determining the severity of pre-eclampsia, the method including measuring the level of a lipidome in a sample isolated from a subject, and comparing the measured level of the lipidome with that of a normal control.

### Solution to Problem

An aspect provides a composition for diagnosing pre-eclampsia, including an agent for measuring the level of a lipidome.

Another aspect provides use of an agent for measuring the level of a lipidome for diagnosing pre-eclampsia.

Another aspect provides use of an agent for measuring the level of a lipidome for preparing a composition for the diagnosis of pre-eclampsia.

In an embodiment, the lipidome may be isolated from one or more selected from the group consisting of tissue, cells, epidermis, blood, plasma, serum, cerebrospinal fluid, bone marrow fluid, lymph, saliva, tears, mucus, urine, amniotic fluid, placenta, and vaginal secretions.

The term “placenta” as used herein refers to an organ that connects the fetus and maternal endometrium and is responsible for functions such as supplying oxygen and nutrients from the mother, protecting the fetus, and excreting waste products from the fetus, and may include the placenta and cord blood.

The term “lipidome” as used herein may include substances produced by metabolism and metabolic processes, substances resulting from chemical metabolism by biological enzymes and molecules, or the like.

As used herein, the term “LPC” means “Lysophosphatidylcholine,” the term “LPE” means “Lysophosphatidylethanolamine,” the term “PC” means “Phosphatidylcholine,” the term “PE” means “Phosphatidylethanolamine,” the term “Plasmenyl PC” means “Plasmenylphosphatidylcholine,” the term “Plasmenyl PE” means “Plasmenylphosphatidylethanolamine,” the term “SM” means “Sphingomyelin,” the term “DAG” means “Diacylglycerol,” the term “TAG” means “Triacylglycerol,” the term “CE” means “Cholesteryl ester,” and the term “Carnitine” means “Fatty Acyl carnitine.”

In an embodiment, the lipidome may be one or more selected from the group consisting of LPE 16:0, LPE 18:0, PE 36:3, PE 38:2, PE 38:1, Plasmenyl PC 34:2, Plasmenyl PC 36:4, Plasmenyl PC 38:6, Plasmenyl PE 36:2(P-18:0/18:2), Plasmenyl PE 40:7 (P-18:1/22:6), Plasmenyl PE 40:6 (P-18:0/22:6), SM 34:2, SM 36:2, SM 42:3, DAG 34:2 (16:1/18:1), DAG 34:1 (18:1/16:0), DAG 34:1 (18:0/16:1), DAG 36:3 (18:1/18:2) and DAG 36:2 (18:1/18:1).

The term “measuring the level” as used herein may include measuring the expression or activity level of a marker for pre-eclampsia-related diseases in a biological sample to diagnose pre-eclampsia-related diseases.

More specifically, the term “measuring the level of a lipidome” as used herein may mean measuring the level of the lipidome, and may be performed by one or more selected from the group consisting of chromatography, liquid chromatography (LC), gas chromatography (GC), mass spectrometry (MS), nuclear magnetic resonance spectroscopy (NMR), thin-layer chromatography (TLC), high performance liquid chromatography (HPLC), ultra (high) performance liquid chromatography (U(H)PLC), gas chromatography-mass spectrometry (GC-MS), liquid chromatography-mass spectrometry (LC-MS), matrix-assisted laser desorption/ionization-MS (MALDI-MS), MALDI-time-of-flight mass spectrometry (MALDI-TOF-MS), tandem mass spectrometry (MS/MS), high performance liquid chromatography-mass spectrometry (HPLC-MS), gas chromatography-time-of-flight-mass spectrometry (GC-TOF-MS), liquid chromatography-time-of-flight-mass spectrometry (LC-TOF-MS), gas chromatography-tandem mass spectrometry (GC-MS/MS), liquid chromatography-tandem mass spectrometry (LC-MS/MS), ultra-performance liquid chromatography-mass spectrometry (UPLC-MS), direct-infusion-MS (DI-MS), ion mobility-MS (IM-MS), electrospray ionization-tandem mass spectrometry (ESI-MS-MS), and mass spectrometry imaging (MSI). However, the present disclosure is not limited thereto.

In an embodiment, the agent for measuring the level of a lipidome may include the agent used in, but is not limited to, one or more selected from the group consisting of chromatography, liquid chromatography, gas chromatography, a mass spectrometer, a nuclear magnetic resonance spectrometer, thin-layer chromatography, high performance liquid chromatography, ultra-high performance liquid chromatography, a gas chromatography-mass spectrometer, a liquid chromatography-mass spectrometer, a matrix-assisted laser desorption/ionization-mass spectrometer, a matrix-assisted laser desorption/ionization-time-of-flight mass spectrometer, a tandem mass spectrometer, a high performance liquid chromatography-mass spectrometer, a gas chromatography-time-of-flight-mass spectrometer, a liquid chromatography-time-of-flight-mass spectrometer, a gas chromatography-tandem mass spectrometer, a liquid chromatography-tandem mass spectrometer, an ultra-high performance liquid chromatography-tandem mass spectrometer, a direct infusion-mass spectrometer, an ion mobility mass spectrometer, an electrospray ionization-tandem mass spectrometer, and a mass spectrometry imaging system.

The term “marker” or “biomarker” as used herein refers to a substance capable of distinguishing between and evaluating subjects with pre-eclampsia and subjects without pre-eclampsia, and the marker or the biomarker may include organic biomolecules that show an increase or decrease in the subjects with pre-eclampsia compared to the subjects without pre-eclampsia, such as polypeptides, nucleic acids (e.g., mRNA and the like), lipids, glycolipids, glycoproteins, sugars (monosaccharides, disaccharides, oligosaccharides, and the like), and lipidomes.

The term “diagnosis” as used herein may refer to determining a subject's susceptibility to a particular disease or condition, determining whether a subject has a particular disease or condition, determining a prognosis of a subject with a particular disease or condition, or monitoring this.

The term “pre-eclampsia (PE)” as used herein refers to a hypertensive disorder complicating pregnancy, and specifically, may be a concept that includes gestational hypertension, and pre-eclampsia and eclampsia, which are more advanced forms of the condition. Symptoms of pre-eclampsia include headache, swelling, nausea, vomiting, foamy urine, and the like. Pre-eclampsia is classified into severe pre-eclampsia (SPE) and non-severe pre-eclampsia (NPE) according to the severity of the symptoms.

In an embodiment, the pathological condition of the pre-eclampsia may be one or more selected from the group consisting of chronic hypertension, gestational hypertension, pre-eclampsia, eclampsia, or complicated pre-eclampsia.

Another aspect provides a composition for determining the severity of pre-eclampsia, including an agent for measuring the level of a lipidome.

Another aspect provides use of an agent for measuring the level of a lipidome for determining the severity of pre-eclampsia.

Another aspect provides use of an agent for preparing a composition for determining the severity of pre-eclampsia, the agent being for measuring the level of a lipidome.

In an embodiment, the lipidome may be isolated from one or more selected from the group consisting of tissue, cells, epidermis, blood, plasma, serum, cerebrospinal fluid, bone marrow fluid, lymph, saliva, tears, mucus, urine, amniotic fluid, placenta, and vaginal secretions.

The placenta, lipidome, level measurement, measurement of the level of a lipidome, agent, and pre-eclampsia are as described above.

In an embodiment, the composition for determining the severity of pre-eclampsia may be a composition for diagnosing severe pre-eclampsia.

In an embodiment, the lipidome may be one or more selected from the group consisting of LPC 16:0, LPC 18:2, LPC 18:0, LPE 18:1, PC 32:0, PC 34:2, PC 34:0, PC 36:5, PC 36:4, PC 36:3, PC 38:6, PC 38:5, PC 38:4, PC 38:3, PC 40:8, PC 40:3, PC 40:2, PC 42:3, PC 42:2, PE 32:0, PE 34:2, PE 34:1, PE 34:0, PE 36:4, PE 36:2, PE 36:1, PE 38:6, PE 38:5, PE 38:4, PE 40:8, PE 40:7, PE 40:6, PE 40:4, PE 40:3, PE 40:2, Plasmenyl PC 32:2, Plasmenyl PC 32:0, Plasmenyl PC 34:1, Plasmenyl PC 36:3, Plasmenyl PC 38:5, Plasmenyl PC 38:4, Plasmenyl PC 38:1, Plasmenyl PC 40:5, Plasmenyl PE 34:2 (P-16:0/18:2), Plasmenyl PE 34:1 (P-16:0/18:1), Plasmenyl PE 36:5 (P-16:0/20:5), Plasmenyl PE 36:4 (P-16:0/20:4), Plasmenyl PE 36:3 (P-16:0/20:3), Plasmenyl PE 38:6 (P-16:0/22:6), Plasmenyl PE 38:5 (P-18:0/20:5), Plasmenyl PE 38:4 (P-18:0/20:4), Plasmenyl PE 38:4 (P-16:0/22:4), Plasmenyl PE 38:3 (P-18:0/20:3), Plasmenyl PE 40:4 (P-20:0/20:4), Plasmenyl PE 40:4 (P-18:0/22:4), Ceramide 34:1 (d18:1/16:0), Ceramide 38:1 (d18:1/20:0), Ceramide 40:1 (d18:1/22:0), Ceramide 42:1 (d18:1/24:0), Ceramide 42:2 (d18:1/24:1), SM 34:1, SM 40:3, SM 40:2, DAG 38:4 (18:0/20:4), DAG 38:3 (18:0/20:3), TAG 46:0 (16:0/14:0/16:0), TAG 48:2 (14:0/18:2/16:0), TAG 48:1 (18:1/14:0/16:0), TAG 48:0 (16:0/16:0/16:0), TAG 50:4 (18:2/14:0/18:2), TAG 50:3 (18:2/16:1/16:0), TAG 50:2 (18:1/16:1/16:0), TAG 50:1 (18:1/16:0/16:0), TAG 50:0 (16:0/18:0/16:0), TAG 51:4 (18:1/15:1/18:2), TAG 51:4 (15:1/18:1/18:2), TAG 51:1 (18:1/17:0/16:0), TAG 52:5 (16:1/18:1/18:3), TAG 52:4 (16:0/18:2/18:2), TAG 52:3 (16:0/18:2/18:1), TAG 52:2 (18:1/16:0/18:1), TAG 52:1 (18:1/18:0/16:0), TAG 54:6 (20:4/16:0/18:2), TAG 54:5 (18:2/18:1/18:2), TAG 54:4 (18:1/18:2/18:1), TAG 54:3 (18:2/18:0/18:1), TAG 54:2 (18:1/18:0/18:1), TAG 54:1 (18:0/18:1/18:0), TAG 56:8, TAG 56:7 (20:1/18:1/18:2), TAG 56:5 (18:1/20:3/18:1), TAG 56:4 (18:2/20:2/18:2), TAG 56:2 (18:1/20:0/18:1), CE 16:1, CE 16:0, CE 18:3, CE 18:2, CE 18:1, CE 18:0, CE 20:5, CE 20:3, CE 20:2, CE 20:1, CE 22:6, CE 22:5, CE 22:4, Carnitine 16:1, Carnitine 16:0, Carnitine 18:2, and Carnitine 18:0.

In an embodiment, the composition for determining the severity of pre-eclampsia may be a composition for diagnosing non-severe pre-eclampsia.

In an embodiment, the lipidome may be one or more selected from the group consisting of LPC 20:4, LPC 20:3, LPE 20:4, LPE 22:6, PE 40:5, Plasmenyl PC 36:5, Ceramide 36:1 (d18:1/18:0), SM 32:1, SM 36:1, SM 38:4, SM 38:1, DAG 32:0 (16:0/16:0), DAG 36:1 (18:0/18:1), TAG 46:1 (14:0/16:1/16:0), and TAG 46:1 (14:0/18:1/14:0).

Another aspect provides a kit for diagnosing pre-eclampsia, including the composition.

The pre-eclampsia is as described above.

In an embodiment, the kit may be, but is not limited to, an integrated magneto-electrochemical sensor kit. An integrated magneto-electrochemical sensor refers to a system that detects an electrical signal by enzymatic signal amplification using magnetic beads. Specifically, the integrated magneto-electrochemical sensor may refer to a system that attaches a substance capable of detecting a target substance (e.g., an antibody) to magnetic beads, and detects an electrical signal generated, through enzymatic signal amplification, by a chromogenic substance capable of detecting the target substance (e.g., horseradish peroxidase (HRP), alkaline phosphatase (ALP), α-D-galactosidase (α-Gal)).

In an embodiment, the kit may include the agent for measuring the level of a lipidome, a device, and a computer having an algorithm built in, and may relate to a kit that associates the result of measuring the level of the marker with the diagnosis or severity determination of pre-eclampsia through the algorithm.

Another aspect provides a kit for determining the severity of pre-eclampsia, including the composition.

The pre-eclampsia and the kit are as described above.

In an embodiment, the kit for determining the severity of pre-eclampsia may be a kit for diagnosing severe pre-eclampsia or non-severe pre-eclampsia.

Another aspect provides a method of diagnosing pre-eclampsia, the method including: measuring the level of a lipidome in a sample isolated from a subject; and comparing the measured level of the lipidome with that of a normal control.

Another aspect provides a method of providing information for the diagnosis of pre-eclampsia, the method including: measuring the level of a lipidome in a sample isolated from a subject; and comparing the measured level of the lipidome with that of a normal control.

In an embodiment, the sample may be one or more selected from the group consisting of tissue, cells, epidermis, blood, plasma, serum, cerebrospinal fluid, bone marrow fluid, lymph, saliva, tears, mucus, urine, amniotic fluid, placenta, and vaginal secretions.

The placenta, lipidome, measurement of the level, measurement of the level of a lipidome, pre-eclampsia, and diagnosis are as described above.

In an embodiment, the measurement of the level of a lipidome may be performed by one or more selected from the group consisting of chromatography, liquid chromatography (LC), gas chromatography (GC), mass spectrometry (MS), nuclear magnetic resonance spectroscopy (NMR), thin-layer chromatography (TLC), high performance liquid chromatography (HPLC), ultra (high) performance liquid chromatography (U(H)PLC), gas chromatography-mass spectrometry (GC-MS), liquid chromatography-mass spectrometry (LC-MS), matrix-assisted laser desorption/ionization-MS (MALDI-MS), MALDI-time-of-flight mass spectrometry (MALDI-TOF-MS), tandem mass spectrometry (MS/MS), high performance liquid chromatography-mass spectrometry (HPLC-MS), gas chromatography-time-of-flight-mass spectrometry (GC-TOF-MS), liquid chromatography-time-of-flight-mass spectrometry (LC-TOF-MS), gas chromatography-tandem mass spectrometry (GC-MS/MS), liquid chromatography-tandem mass spectrometry (LC-MS/MS), ultra-performance liquid chromatography-mass spectrometry (UPLC-MS), direct-infusion-MS (DI-MS), ion mobility-MS (IM-MS), electrospray ionization-tandem mass spectrometry (ESI-MS-MS), and mass spectrometry imaging (MSI). However, the present disclosure is not limited thereto.

In an embodiment, the lipidome may be one or more selected from the group consisting of LPE 16:0, LPE 18:0, PE 36:3, PE 38:2, PE 38:1, Plasmenyl PC 34:2, Plasmenyl PC 36:4, Plasmenyl PC 38:6, Plasmenyl PE 36:2 (P-18:0/18:2), Plasmenyl PE 40:7 (P-18:1/22:6), Plasmenyl PE 40:6 (P-18:0/22:6), SM 34:2, SM 36:2, SM 42:3, DAG 34:2 (16:1/18:1), DAG 34:1 (18:1/16:0), DAG 34:1 (18:0/16:1), DAG 36:3 (18:1/18:2), and DAG 36:2 (18:1/18:1).

In an embodiment, the method of diagnosing pre-eclampsia may further include determining that, in case that the level of one or more lipidomes selected from the group consisting of LPE 16:0, LPE 18:0, PE 36:3, PE 38:2, PE 38:1, Plasmenyl PC 34:2, Plasmenyl PC 36:4, Plasmenyl PC 38:6, Plasmenyl PE 36:2 (P-18:0/18:2), Plasmenyl PE 40:7 (P-18:1/22:6), Plasmenyl PE 40:6 (P-18:0/22:6), SM 34:2, SM 36:2, and SM 42:3 is decreased compared to the control, or in case that the level of one or more lipidomes selected from the group consisting of DAG 34:2 (16:1/18:1), DAG 34:1 (18:1/16:0), DAG 34:1 (18:0/16:1), DAG 36:3 (18:1/18:2), and DAG 36:2 (18:1/18:1) is increased compared to the control, the subject is diagnosed as having pre-eclampsia.

The term “decrease in the level of a lipidome” as used herein means a measurable and significant decrease in the concentration of a lipidome in a sample of a subject compared to a control, for example, a decrease to 0.9-fold or less relative to the control, for example, by 0.1-fold to 0.9-fold, 0.9-fold, 0.8-fold, 0.7-fold, 0.6-fold, 0.5-fold, 0.4-fold, 0.3-fold, 0.2-fold, or 0.1-fold or less.

The term “increase in the level of a lipidome” as used herein means a measurable and significant increase in the concentration of a lipidome in a sample of a subject compared to a control, for example, an increase by about 1.1-fold relative to the control or more, for example, by 1.1-fold to 2.5-fold, 1.1-fold, 1.2-fold, 1.3-fold, 1.4-fold, 1.5-fold, 1.6-fold, 1.7-fold, 1.8-fold, 1.9-fold, or 2-fold or more.

In an embodiment, the method of diagnosing pre-eclampsia may be performed using mass panels.

Another aspect provides a method of determining the severity of pre-eclampsia, the method including: measuring the level of a lipidome in a sample isolated from a subject; and comparing the measured level of the lipidome with that of a normal control.

Another aspect provides a method of providing information for determining the severity of pre-eclampsia, the method including: measuring the level of a lipidome in a sample isolated from a subject; and comparing the measured level of the lipidome with that of a normal control.

In an embodiment, the sample may be one or more selected from the group consisting of tissue, cells, epidermis, blood, plasma, serum, cerebrospinal fluid, bone marrow fluid, lymph, saliva, tears, mucus, urine, amniotic fluid, placenta, and vaginal secretions.

The placenta, lipidome, measurement of the level, measurement of the level of a lipidome, and pre-eclampsia are as described above.

In an embodiment, the measurement of the level of a lipidome may be performed by one or more selected from the group consisting of chromatography, liquid chromatography (LC), gas chromatography (GC), mass spectrometry (MS), nuclear magnetic resonance spectroscopy (NMR), thin-layer chromatography (TLC), high performance liquid chromatography (HPLC), ultra (high) performance liquid chromatography (U(H)PLC), gas chromatography-mass spectrometry (GC-MS), liquid chromatography-mass spectrometry (LC-MS), matrix-assisted laser desorption/ionization-MS (MALDI-MS), MALDI-time-of-flight mass spectrometry (MALDI-TOF-MS), tandem mass spectrometry (MS/MS), high performance liquid chromatography-mass spectrometry (HPLC-MS), gas chromatography-time-of-flight-mass spectrometry (GC-TOF-MS), liquid chromatography-time-of-flight-mass spectrometry (LC-TOF-MS), gas chromatography-tandem mass spectrometry (GC-MS/MS), liquid chromatography-tandem mass spectrometry (LC-MS/MS), ultra-performance liquid chromatography-mass spectrometry (UPLC-MS), direct-infusion-MS (DI-MS), ion mobility-MS (IM-MS), electrospray ionization-tandem mass spectrometry (ESI-MS-MS), and mass spectrometry imaging (MSI). However, the present disclosure is not limited thereto.

In an embodiment, the method of determining the severity of pre-eclampsia may be a method of diagnosing severe pre-eclampsia.

In an embodiment, the lipidome may be one or more selected from the group consisting of LPC 16:0, LPC 18:2, LPC 18:0, LPE 18:1, PC 32:0, PC 34:2, PC 34:0, PC 36:5, PC 36:4, PC 36:3, PC 38:6, PC 38:5, PC 38:4, PC 38:3, PC 40:8, PC 40:3, PC 40:2, PC 42:3, PC 42:2, PE 32:0, PE 34:2, PE 34:1, PE 34:0, PE 36:4, PE 36:2, PE 36:1, PE 38:6, PE 38:5, PE 38:4, PE 40:8, PE 40:7, PE 40:6, PE 40:4, PE 40:3, PE 40:2, Plasmenyl PC 32:2, Plasmenyl PC 32:0, Plasmenyl PC 34:1, Plasmenyl PC 36:3, Plasmenyl PC 38:5, Plasmenyl PC 38:4, Plasmenyl PC 38:1, Plasmenyl PC 40:5, Plasmenyl PE 34:2 (P-16:0/18:2), Plasmenyl PE 34:1 (P-16:0/18:1), Plasmenyl PE 36:5 (P-16:0/20:5), Plasmenyl PE 36:4 (P-16:0/20:4), Plasmenyl PE 36:3 (P-16:0/20:3), Plasmenyl PE 38:6 (P-16:0/22:6), Plasmenyl PE 38:5 (P-18:0/20:5), Plasmenyl PE 38:4 (P-18:0/20:4), Plasmenyl PE 38:4 (P-16:0/22:4), Plasmenyl PE 38:3 (P-18:0/20:3), Plasmenyl PE 40:4 (P-20:0/20:4), Plasmenyl PE 40:4 (P-18:0/22:4), Ceramide 34:1 (d18:1/16:0), Ceramide 38:1 (d18:1/20:0), Ceramide 40:1 (d18:1/22:0), Ceramide 42:1 (d18:1/24:0), Ceramide 42:2 (d18:1/24:1), SM 34:1, SM 40:3, SM 40:2, DAG 38:4 (18:0/20:4), DAG 38:3 (18:0/20:3), TAG 46:0 (16:0/14:0/16:0), TAG 48:2 (14:0/18:2/16:0), TAG 48:1 (18:1/14:0/16:0), TAG 48:0 (16:0/16:0/16:0), TAG 50:4 (18:2/14:0/18:2), TAG 50:3 (18:2/16:1/16:0), TAG 50:2 (18:1/16:1/16:0), TAG 50:1 (18:1/16:0/16:0), TAG 50:0 (16:0/18:0/16:0), TAG 51:4 (18:1/15:1/18:2), TAG 51:4 (15:1/18:1/18:2), TAG 51:1 (18:1/17:0/16:0), TAG 52:5 (16:1/18:1/18:3), TAG 52:4 (16:0/18:2/18:2), TAG 52:3 (16:0/18:2/18:1), TAG 52:2 (18:1/16:0/18:1), TAG 52:1 (18:1/18:0/16:0), TAG 54:6 (20:4/16:0/18:2), TAG 54:5 (18:2/18:1/18:2), TAG 54:4 (18:1/18:2/18:1), TAG 54:3 (18:2/18:0/18:1), TAG 54:2 (18:1/18:0/18:1), TAG 54:1 (18:0/18:1/18:0), TAG 56:8, TAG 56:7 (20:1/18:1/18:2), TAG 56:5 (18:1/20:3/18:1), TAG 56:4 (18:2/20:2/18:2), TAG 56:2 (18:1/20:0/18:1), CE 16:1, CE 16:0, CE 18:3, CE 18:2, CE 18:1, CE 18:0, CE 20:5, CE 20:3, CE 20:2, CE 20:1, CE 22:6, CE 22:5, CE 22:4, Carnitine 16:1, Carnitine 16:0, Carnitine 18:2, and Carnitine 18:0.

In an embodiment, the method of diagnosing severe pre-eclampsia may further include determining that, in case that the level of one or more lipidomes selected from the group consisting of LPC 16:0, LPC 18:2, LPC 18:0, LPE 18:1, PC 32:0, PC 34:2, PC 34:0, PC 36:5, PC 36:4, PC 36:3, PC 38:6, PC 38:5, PC 38:4, PC 38:3, PC 40:8, PC 40:3, PC 40:2, PC 42:3, PC 42:2, PE 32:0, PE 34:2, PE 34:1, PE 34:0, PE 36:4, PE 36:2, PE 36:1, PE 38:6, PE 38:5, PE 38:4, PE 40:8, PE 40:7, PE 40:6, PE 40:4, PE 40:3, PE 40:2, Plasmenyl PC 32:2, Plasmenyl PC 32:0, Plasmenyl PC 34:1, Plasmenyl PC 36:3, Plasmenyl PC 38:5, Plasmenyl PC 38:4, Plasmenyl PC 38:1, Plasmenyl PC 40:5, Plasmenyl PE 34:2 (P-16:0/18:2), Plasmenyl PE 34:1 (P-16:0/18:1), Plasmenyl PE 36:5 (P-16:0/20:5), Plasmenyl PE 36:4 (P-16:0/20:4), Plasmenyl PE 36:3 (P-16:0/20:3), Plasmenyl PE 38:6 (P-16:0/22:6), Plasmenyl PE 38:5 (P-18:0/20:5), Plasmenyl PE 38:4 (P-18:0/20:4), Plasmenyl PE 38:4 (P-16:0/22:4), Plasmenyl PE 38:3 (P-18:0/20:3), Plasmenyl PE 40:4 (P-20:0/20:4), Plasmenyl PE 40:4 (P-18:0/22:4), Ceramide 34:1 (d18:1/16:0), Ceramide 38:1 (d18:1/20:0), Ceramide 40:1 (d18:1/22:0), Ceramide 42:1 (d18:1/24:0), Ceramide 42:2 (d18:1/24:1), SM 34:1, SM 40:3, SM 40:2, DAG 38:4 (18:0/20:4), DAG 38:3 (18:0/20:3), Carnitine 16:1, Carnitine 16:0, Carnitine 18:2, and Carnitine 18:0 is decreased compared to the control, or in case that the level of one or more lipidomes selected from the group consisting of TAG 46:0 (16:0/14:0/16:0), TAG 48:2 (14:0/18:2/16:0), TAG 48:1 (18:1/14:0/16:0), TAG 48:0 (16:0/16:0/16:0), TAG 50:4 (18:2/14:0/18:2), TAG 50:3 (18:2/16:1/16:0), TAG 50:2 (18:1/16:1/16:0), TAG 50:1 (18:1/16:0/16:0), TAG 50:0 (16:0/18:0/16:0), TAG 51:4 (18:1/15:1/18:2), TAG 51:4 (15:1/18:1/18:2), TAG 51:1 (18:1/17:0/16:0), TAG 52:5 (16:1/18:1/18:3), TAG 52:4 (16:0/18:2/18:2), TAG 52:3 (16:0/18:2/18:1), TAG 52:2 (18:1/16:0/18:1), TAG 52:1 (18:1/18:0/16:0), TAG 54:6 (20:4/16:0/18:2), TAG 54:5 (18:2/18:1/18:2), TAG 54:4 (18:1/18:2/18:1), TAG 54:3 (18:2/18:0/18:1), TAG 54:2 (18:1/18:0/18:1), TAG 54:1 (18:0/18:1/18:0), TAG 56:8, TAG 56:7 (20:1/18:1/18:2), TAG 56:5 (18:1/20:3/18:1), TAG 56:4 (18:2/20:2/18:2), TAG 56:2 (18:1/20:0/18:1), CE 16:1, CE 16:0, CE 18:3, CE 18:2, CE 18:1, CE 18:0, CE 20:5, CE 20:3, CE 20:2, CE 20:1, CE 22:6, CE 22:5, and CE 22:4 is increased compared to the control, the subject is diagnosed as having severe pre-eclampsia.

The decrease in the level of a lipidome and the increase in the level of a lipidome are as described above.

In an embodiment, the method of diagnosing severe pre-eclampsia may be performed using mass panels.

In an embodiment, the method of determining the severity of pre-eclampsia may be a method of diagnosing non-severe pre-eclampsia.

In an embodiment, the lipidome may be one or more selected from the group consisting of LPC 20:4, LPC 20:3, LPE 20:4, LPE 22:6, PE 40:5, Plasmenyl PC 36:5, Ceramide 36:1 (d18:1/18:0), SM 32:1, SM 36:1, SM 38:4, SM 38:1, DAG 32:0 (16:0/16:0), DAG 36:1 (18:0/18:1), TAG 46:1 (14:0/16:1/16:0), and TAG 46:1 (14:0/18:1/14:0).

In an embodiment, the method of diagnosing non-severe pre-eclampsia may further include determining that, in case that the level of one or more lipidomes selected from the group consisting of LPC 20:4, LPC 20:3, LPE 20:4, LPE 22:6, PE 40:5, Ceramide 36:1 (d18:1/18:0), SM 36:1, SM 38:1, DAG 32:0 (16:0/16:0), and DAG 36:1 (18:0/18:1) is increased compared to the control, or in case that the level of one or more lipidomes selected from the group consisting of Plasmenyl PC 36:5, SM 32:1, SM 38:4, TAG 46:1 (14:0/16:1/16:0), and TAG 46:1 (14:0/18:1/14:0) is decreased compared to the control, the subject is diagnosed as having non-severe pre-eclampsia.

The decrease in the level of a lipidome and the increase in the level of a lipidome are as described above.

In an embodiment, the method of diagnosing non-severe pre-eclampsia may be performed using mass panels.

### Advantageous Effects of Invention

A composition for diagnosing pre-eclampsia and a composition for determining the severity of pre-eclampsia, according to an aspect, can diagnose pre-eclampsia and significantly determine the severity of the disease, by measuring the level of a lipidome that changes in patients and comparing the measured level.

### BRIEF DESCRIPTION OF DRAWINGS

FIG. 1 illustrates the results of heatmap analysis of the levels of biomarkers selected for the diagnosis of pre-eclampsia.

FIGS. 2 to 4 illustrates the results of heatmap analysis of the levels of biomarkers selected for the diagnosis of severe pre-eclampsia.

FIG. 5 illustrates the results of heatmap analysis of the levels of biomarkers selected for the diagnosis of non-severe pre-eclampsia.

### MODE FOR THE INVENTION

Hereinafter, preferred examples will be presented to aid in understanding of the present disclosure. However, the following examples are provided only for the purpose of easier understanding of the present disclosure, and the present disclosure is not limited by the following examples. As embodiments may allow for various modifications, the embodiments are not limited to the examples disclosed hereinafter but may be embodied in various forms.

The terms or words used in the specification and the claims of the present disclosure should not be construed as being limited to ordinary or dictionary meanings and should be construed as meanings and concepts consistent with the technical idea of the present disclosure based on a principle that an inventor can appropriately define concepts of terms to explain his/her invention in the best way.

Throughout the specification of the present disclosure, when a part is referred to as “including” a certain element, it means a case where the part may further include other elements, rather than precluding other elements, unless otherwise specifically stated herein.

In the entire specification of the present disclosure, “A and/or B” means A or B, or A and B.

### Example 1. Placental Lipidome Extraction

Lipidomes were extracted from placental samples of 9 normal pregnant women and 12 pre-eclampsia cases (7 cases of severe pre-eclampsia and 5 cases of non-severe pre-eclampsia). 300 mL of 100% MeOH containing 0.1% BHT and 100 mL of distilled water were added to 40 mg of placenta and mixed together for 1 minute, and the mixture was homogenized by using a Mixermill 4 times for 30 seconds. Subsequently, stainless beads were removed, and 1 mL of MTBE containing 0.1% BHT was added and mixed at room temperature for one hour. 100 mL of distilled water was added and mixed at room temperature for 10 minutes, and then the mixture was centrifuged twice at 14,000 rpm for 15 minutes. 220 mL of the supernatant and 110 mL of the infranatant were taken and concentrated through a speed vac concentrator, and then the dried samples were re-dissolved using 80 μL of CHCl3:MeOH (1:9) and 20 μL of IS.

### Example 2. Selection of Biomarkers for Diagnosis of Pre-Eclampsia

To select biomarkers for the diagnosis of pre-eclampsia, placental lipidomes were extracted, followed by lipidomic analysis using LC-MS/MS. The previously re-dissolved samples were transferred to auto sampler vials, and then analyzed by LC-MS/MS. LC-MS/MS was performed using an LC system consisting of a Nexera X2 binary pump system (LC-30 AD, Shimadzu), an auto-sampler (CTO-20AC Autosampler), a mass spectrometer (LCMS 8060, Triple quadrupole mass spectrometer system: Shimadzu), and a column (Kinetex C18 Column: 2.6 μm, 2.1 mm×100 mm), under conditions of a 5 μL injection volume, mobile phase: solvent A=methanol/water (90/10+10 mM ammonium acetate), and solvent B=methanol/isopropanol (50/50+10 mM ammonium acetate).

Analysis was performed under lipid-specific selected reaction monitoring conditions for a total of 244 lipids, and each peak was integrated using the Lab Solutions program. Thereafter, one-point calibration was conducted based on an internal standard material added to the analytical samples to quantify each lipid component. Thereafter, multivariate statistical analysis was performed using SIMCA-P+ (Ver. 12.0) software to select disease-specific lipidomes. The criterion was p value<0.05. Lipids showing final content changes were selected as biomarkers for the diagnosis of pre-eclampsia.

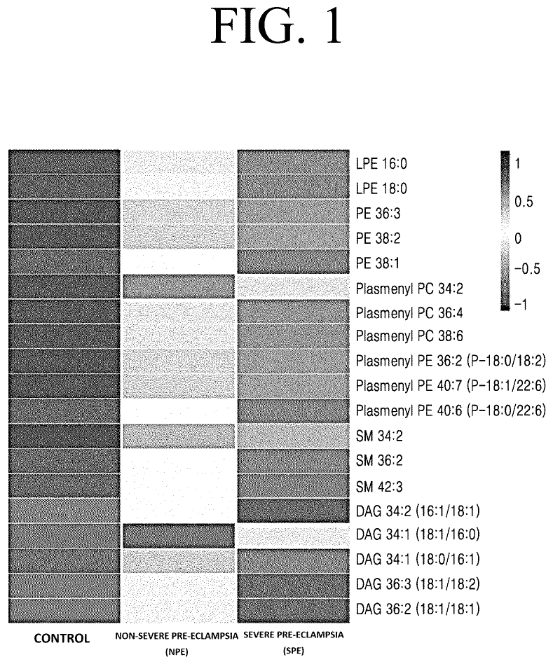

FIG. 1 illustrates the results of heatmap analysis of the levels of biomarkers selected for the diagnosis of pre-eclampsia.

As illustrated in FIG. 1, relatively low levels of LPE 16:0, LPE 18:0, PE 36:3, PE 38:2, PE 38:1, Plasmenyl PC 34:2, Plasmenyl PC 36:4, Plasmenyl PC 38:6, Plasmenyl PE 36:2 (P-18:0/18:2), Plasmenyl PE 40:7 (P-18:1/22:6), Plasmenyl PE 40:6 (P-18:0/22:6), SM 34:2, SM 36:2, and SM 42:3 were shown in a pre-eclampsia group compared to a control, and
relatively high levels of DAG 34:2 (16:1/18:1), DAG 34:1 (18:1/16:0), DAG 34:1 (18:0/16:1), DAG 36:3 (18:1/18:2), and DAG 36:2 (18:1/18:1) were shown in the pre-eclampsia group compared to the control.

### Example 3. Selection of Biomarkers for Diagnosis of Severe Pre-Eclampsia

To select biomarkers for the diagnosis of severe pre-eclampsia, placental lipidomes were extracted, followed by lipidomic analysis using LC-MS/MS.

The lipidomic analysis method and the multivariate statistical analysis were performed in the same manner as in Example 2. As a result of performance, metabolites showing final expression changes were selected as biomarkers for diagnosing severe pre-eclampsia.

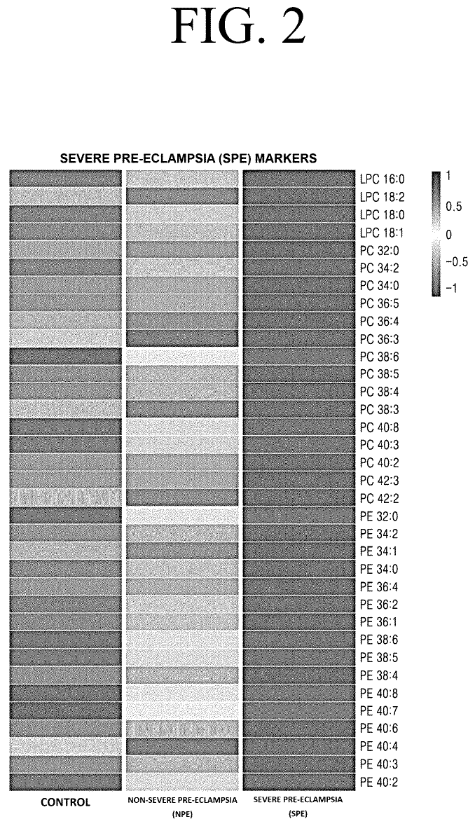

FIGS. 2 to 4 illustrates the results of heatmap analysis of the levels of biomarkers selected for the diagnosis of severe pre-eclampsia.

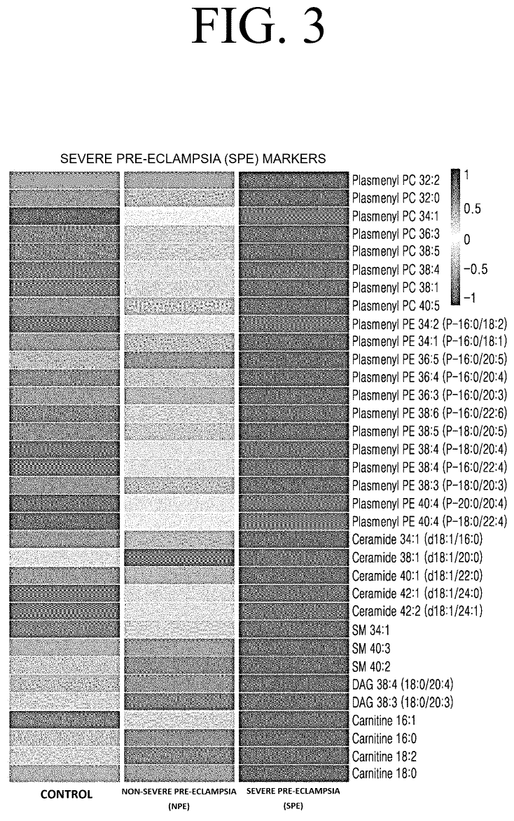

As illustrated in FIGS. 2 and 3, relatively low levels of LPC 16:0, LPC 18:2, LPC 18:0, LPE 18:1, PC 32:0, PC 34:2, PC 34:0, PC 36:5, PC 36:4, PC 36:3, PC 38:6, PC 38:5, PC 38:4, PC 38:3, PC 40:8, PC 40:3, PC 40:2, PC 42:3, PC 42:2, PE 32:0, PE 34:2, PE 34:1, PE 34:0, PE 36:4, PE 36:2, PE 36:1, PE 38:6, PE 38:5, PE 38:4, PE 40:8, PE 40:7, PE 40:6, PE 40:4, PE 40:3, PE 40:2, Plasmenyl PC 32:2, Plasmenyl PC 32:0, Plasmenyl PC 34:1, Plasmenyl PC 36:3, Plasmenyl PC 38:5, Plasmenyl PC 38:4, Plasmenyl PC 38:1, Plasmenyl PC 40:5, Plasmenyl PE 34:2 (P-16:0/18:2), Plasmenyl PE 34:1 (P-16:0/18:1), Plasmenyl PE 36:5 (P-16:0/20:5), Plasmenyl PE 36:4 (P-16:0/20:4), Plasmenyl PE 36:3 (P-16:0/20:3), Plasmenyl PE 38:6 (P-16:0/22:6), Plasmenyl PE 38:5 (P-18:0/20:5), Plasmenyl PE 38:4 (P-18:0/20:4), Plasmenyl PE 38:4 (P-16:0/22:4), Plasmenyl PE 38:3 (P-18:0/20:3), Plasmenyl PE 40:4 (P-20:0/20:4), Plasmenyl PE 40:4 (P-18:0/22:4), Ceramide 34:1 (d18:1/16:0), Ceramide 38:1 (d18:1/20:0), Ceramide 40:1 (d18:1/22:0), Ceramide 42:1 (d18:1/24:0), Ceramide 42:2 (d18:1/24:1), SM 34:1, SM 40:3, SM 40:2, DAG 38:4 (18:0/20:4), DAG 38:3 (18:0/20:3), Carnitine 16:1, Carnitine 16:0, Carnitine 18:2, and Carnitine 18:0 were shown in a severe pre-eclampsia group compared to a control and a non-severe pre-eclampsia group.

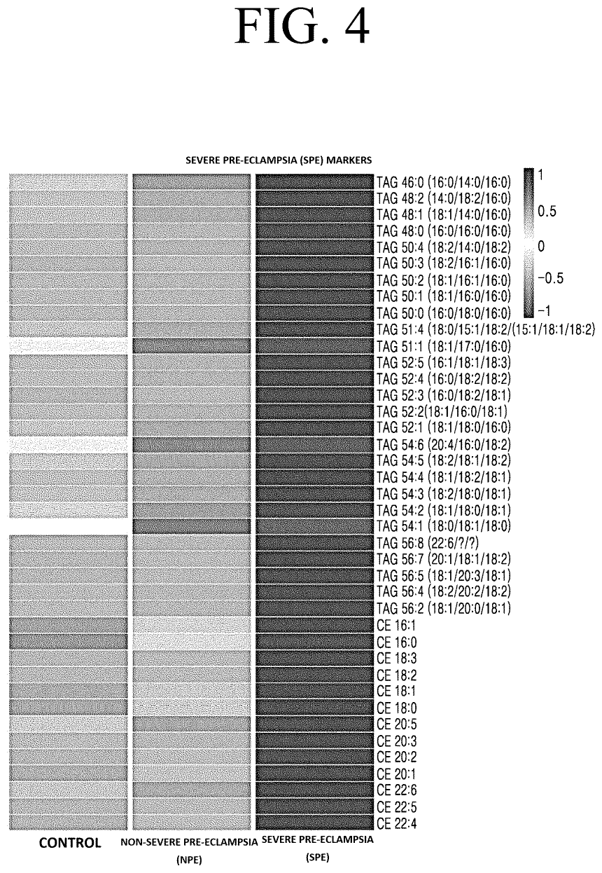

As illustrated in FIG. 4, relatively high levels of TAG 46:0 (16:0/14:0/16:0), TAG 48:2 (14:0/18:2/16:0), TAG 48:1 (18:1/14:0/16:0), TAG 48:0 (16:0/16:0/16:0), TAG 50:4 (18:2/14:0/18:2), TAG 50:3 (18:2/16:1/16:0), TAG 50:2 (18:1/16:1/16:0), TAG 50:1 (18:1/16:0/16:0), TAG 50:0 (16:0/18:0/16:0), TAG 51:4 (18:1/15:1/18:2), TAG 51:4 (15:1/18:1/18:2), TAG 51:1 (18:1/17:0/16:0), TAG 52:5 (16:1/18:1/18:3), TAG 52:4 (16:0/18:2/18:2), TAG 52:3 (16:0/18:2/18:1), TAG 52:2 (18:1/16:0/18:1), TAG 52:1 (18:1/18:0/16:0), TAG 54:6 (20:4/16:0/18:2), TAG 54:5 (18:2/18:1/18:2), TAG 54:4 (18:1/18:2/18:1), TAG 54:3 (18:2/18:0/18:1), TAG 54:2 (18:1/18:0/18:1), TAG 54:1 (18:0/18:1/18:0), TAG 56:8, TAG 56:7 (20:1/18:1/18:2), TAG 56:5 (18:1/20:3/18:1), TAG 56:4(18:2/20:2/18:2), TAG 56:2 (18:1/20:0/18:1), CE 16:1, CE 16:0, CE 18:3, CE 18:2, CE 18:1, CE 18:0, CE 20:5, CE 20:3, CE 20:2, CE 20:1, CE 22:6, CE 22:5, and CE 22:4 were shown in a severe pre-eclampsia group compared to a control and a non-severe pre-eclampsia group.

### Example 4. Selection of Biomarkers for Diagnosis of Non-Severe Pre-Eclampsia

To select biomarkers for the diagnosis of non-severe pre-eclampsia, placental lipidomes were extracted, followed by lipidomic analysis using LC-MS/MS.

The lipidomic analysis method and the multivariate statistical analysis were performed in the same manner as in Example 2. As a result of performance, metabolites showing final expression changes were selected as biomarkers for diagnosing non-severe pre-eclampsia.

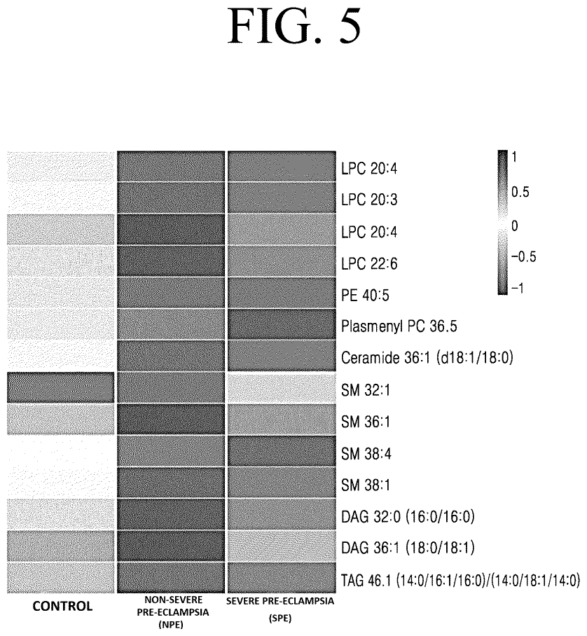

FIG. 5 illustrates the results of heatmap analysis of the levels of biomarkers selected for the diagnosis of non-severe pre-eclampsia.

As illustrated in FIG. 5, relatively high levels of LPC 20:4, LPC 20:3, LPE 20:4, LPE 22:6, PE 40:5, Ceramide 36:1 (d18:1/18:0), SM 36:1, SM 38:1, DAG 32:0 (16:0/16:0), and DAG 36:1 (18:0/18:1) were shown in the non-severe pre-eclampsia group compared to the control and the severe pre-eclampsia group, and
relatively low levels of Plasmenyl PC 36:5, SM 32:1, SM 38:4, TAG 46:1 (14:0/16:1/16:0), and TAG 46:1 (14:0/18:1/14:0) were shown in the non-severe pre-eclampsia group compared to the control and the severe pre-eclampsia group.

From the foregoing description, it will be understood by those of ordinary skill in the art to which the present disclosure pertains that the present disclosure may be embodied in other particular forms without changing the technical spirit or essential characteristics of the present disclosure. Thus, the experimental examples and examples described herein should be considered in an illustrative sense only and not for the purpose of limitation. All changes or modifications derived from the meaning and scope of the appended claims described below rather than the detailed description and equivalent concepts thereof should be construed as falling within the scope of the present disclosure.

1. A method of providing information for diagnosis of pre-eclampsia, the method comprising: measuring a level of a lipidome in a sample isolated from a subject; and comparing the measured level of the lipidome with that of a normal control.

2. The method of claim 1, wherein the sample is one or more selected from the group consisting of tissue, cells, epidermis, blood, plasma, serum, cerebrospinal fluid, bone marrow fluid, lymph, saliva, tears, mucus, urine, amniotic fluid, placenta, and vaginal secretions.

3. The method of claim 1, wherein the lipidome is one or more selected from the group consisting of LPE 16:0, LPE 18:0, PE 36:3, PE 38:2, PE 38:1, Plasmenyl PC 34:2, Plasmenyl PC 36:4, Plasmenyl PC 38:6, Plasmenyl PE 36:2 (P-18:0/18:2), Plasmenyl PE 40:7 (P-18:1/22:6), Plasmenyl PE 40:6 (P-18:0/22:6), SM 34:2, SM 36:2, SM 42:3, DAG 34:2 (16:1/18:1), DAG 34:1 (18:1/16:0), DAG 34:1 (18:0/16:1), DAG 36:3 (18:1/18:2), and DAG 36:2 (18:1/18:1).

4. The method of claim 3, further comprising determining that the subject is pre-eclampsia:
in case that the level of one or more lipidomes selected from the group consisting of LPE 16:0, LPE 18:0, PE 36:3, PE 38:2, PE 38:1, Plasmenyl PC 34:2, Plasmenyl PC 36:4, Plasmenyl PC 38:6, Plasmenyl PE 36:2 (P-18:0/18:2), Plasmenyl PE 40:7 (P-18:1/22:6), Plasmenyl PE 40:6 (P-18:0/22:6), SM 34:2, SM 36:2, and SM 42:3 is decreased compared to the control; or
in case that the level of one or more lipidomes selected from the group consisting of DAG 34:2 (16:1/18:1), DAG 34:1 (18:1/16:0), DAG 34:1 (18:0/16:1), DAG 36:3 (18:1/18:2), and DAG 36:2 (18:1/18:1) is increased compared to the control.

5. A method of determining severity of pre-eclampsia, the method comprising: measuring a level of a lipidome in a sample isolated from a subject; and comparing the measured level of the lipidome with that of a normal control.

6. The method of claim 5, wherein the sample is one or more selected from the group consisting of tissue, cells, epidermis, blood, plasma, serum, cerebrospinal fluid, bone marrow fluid, lymph, saliva, tears, mucus, urine, amniotic fluid, placenta, and vaginal secretions.

7. The method of claim 5, wherein the lipidome is one or more selected from the group consisting of LPC 18:2, LPC 18:0, LPE 18:1, PC 32:0, PC 34:2, PC 34:0, PC 36:5, PC 36:4, PC 36:3, PC 38:6, PC 38:5, PC 38:4, PC 38:3, PC 40:8, PC 40:3, PC 40:2, PC 42:3, PC 42:2, PE 32:0, PE 34:2, PE 34:1, PE 34:0, PE 36:4, PE 36:2, PE 36:1, PE 38:6, PE 38:5, PE 38:4, PE 40:8, PE 40:7, PE 40:6, PE 40:4, PE 40:3, PE 40:2, Plasmenyl PC 32:2, Plasmenyl PC 32:0, Plasmenyl PC 34:1, Plasmenyl PC 36:3, Plasmenyl PC 38:5, Plasmenyl PC 38:4, Plasmenyl PC 38:1, Plasmenyl PC 40:5, Plasmenyl PE 34:2 (P-16:0/18:2), Plasmenyl PE 34:1 (P-16:0/18:1), Plasmenyl PE 36:5 (P-16:0/20:5), Plasmenyl PE 36:4 (P-16:0/20:4), Plasmenyl PE 36:3 (P-16:0/20:3), Plasmenyl PE 38:6 (P-16:0/22:6), Plasmenyl PE 38:5 (P-18:0/20:5), Plasmenyl PE 38:4 (P-18:0/20:4), Plasmenyl PE 38:4 (P-16:0/22:4), Plasmenyl PE 38:3 (P-18:0/20:3), Plasmenyl PE 40:4 (P-20:0/20:4), Plasmenyl PE 40:4 (P-18:0/22:4), Ceramide 34:1 (d18:1/16:0), Ceramide 38:1 (d18:1/20:0), Ceramide 40:1 (d18:1/22:0), Ceramide 42:1 (d18:1/24:0), Ceramide 42:2 (d18:1/24:1), SM 34:1, SM 40:3, SM 40:2, DAG 38:4 (18:0/20:4), DAG 38:3 (18:0/20:3), TAG 46:0 (16:0/14:0/16:0), TAG 48:2 (14:0/18:2/16:0), TAG 48:1(18:1/14:0/16:0), TAG 48:0 (16:0/16:0/16:0), TAG 50:4 (18:2/14:0/18:2), TAG 50:3 (18:2/16:1/16:0), TAG 50:2 (18:1/16:1/16:0), TAG 50:1 (18:1/16:0/16:0), TAG 50:0 (16:0/18:0/16:0), TAG 51:4 (18:1/15:1/18:2), TAG 51:4 (15:1/18:1/18:2), TAG 51:1 (18:1/17:0/16:0), TAG 52:5 (16:1/18:1/18:3), TAG 52:4 (16:0/18:2/18:2), TAG 52:3 (16:0/18:2/18:1), TAG 52:2 (18:1/16:0/18:1), TAG 52:1 (18:1/18:0/16:0), TAG 54:6 (20:4/16:0/18:2), TAG 54:5 (18:2/18:1/18:2), TAG 54:4 (18:1/18:2/18:1), TAG 54:3 (18:2/18:0/18:1), TAG 54:2 (18:1/18:0/18:1), TAG 54:1 (18:0/18:1/18:0), TAG 56:8, TAG 56:7 (20:1/18:1/18:2), TAG 56:5 (18:1/20:3/18:1), TAG 56:4 (18:2/20:2/18:2), TAG 56:2 (18:1/20:0/18:1), CE 16:1, CE 16:0, CE 18:3, CE 18:2, CE 18:1, CE 18:0, CE 20:5, CE 20:3, CE 20:2, CE 20:1, CE 22:6, CE 22:5, CE 22:4, Carnitine 16:1, Carnitine 16:0, Carnitine 18:2, and Carnitine 18:0.

8. The method of claim 7, wherein the lipidome further comprises LPC 16:0.

9. The method of determining the severity of pre-eclampsia of claim 7, further comprising determining that the subject is diagnosed as having severe pre-eclampsia:
in case that the level of one or more lipidomes selected from the group consisting of LPC 18:2, LPC 18:0, LPE 18:1, PC 32:0, PC 34:2, PC 34:0, PC 36:5, PC 36:4, PC 36:3, PC 38:6, PC 38:5, PC 38:4, PC 38:3, PC 40:8, PC 40:3, PC 40:2, PC 42:3, PC 42:2, PE 32:0, PE 34:2, PE 34:1, PE 34:0, PE 36:4, PE 36:2, PE 36:1, PE 38:6, PE 38:5, PE 38:4, PE 40:8, PE 40:7, PE 40:6, PE 40:4, PE 40:3, PE 40:2, Plasmenyl PC 32:2, Plasmenyl PC 32:0, Plasmenyl PC 34:1, Plasmenyl PC 36:3, Plasmenyl PC 38:5, Plasmenyl PC 38:4, Plasmenyl PC 38:1, Plasmenyl PC 40:5, Plasmenyl PE 34:2 (P-16:0/18:2), Plasmenyl PE 34:1 (P-16:0/18:1), Plasmenyl PE 36:5 (P-16:0/20:5), Plasmenyl PE 36:4 (P-16:0/20:4), Plasmenyl PE 36:3 (P-16:0/20:3), Plasmenyl PE 38:6 (P-16:0/22:6), Plasmenyl PE 38:5 (P-18:0/20:5), Plasmenyl PE 38:4 (P-18:0/20:4), Plasmenyl PE 38:4 (P-16:0/22:4), Plasmenyl PE 38:3 (P-18:0/20:3), Plasmenyl PE 40:4 (P-20:0/20:4), Plasmenyl PE 40:4 (P-18:0/22:4), Ceramide 34:1 (d18:1/16:0), Ceramide 38:1 (d18:1/20:0), Ceramide 40:1 (d18:1/22:0), Ceramide 42:1 (d18:1/24:0), Ceramide 42:2 (d18:1/24:1), SM 34:1, SM 40:3, SM 40:2, DAG 38:4 (18:0/20:4), DAG 38:3 (18:0/20:3), Carnitine 16:1, Carnitine 16:0, Carnitine 18:2, and Carnitine 18:0 is decreased compared to the control; or
in case that the level of one or more lipidomes selected from the group consisting of TAG 46:0 (16:0/14:0/16:0), TAG 48:2 (14:0/18:2/16:0), TAG 48:1 (18:1/14:0/16:0), TAG 48:0 (16:0/16:0/16:0), TAG 50:4 (18:2/14:0/18:2), TAG 50:3 (18:2/16:1/16:0), TAG 50:2 (18:1/16:1/16:0), TAG 50:1 (18:1/16:0/16:0), TAG 50:0 (16:0/18:0/16:0), TAG 51:4 (18:1/15:1/18:2), TAG 51:4 (15:1/18:1/18:2), TAG 51:1 (18:1/17:0/16:0), TAG 52:5 (16:1/18:1/18:3), TAG 52:4 (16:0/18:2/18:2), TAG 52:3 (16:0/18:2/18:1), TAG 52:2 (18:1/16:0/18:1), TAG 52:1 (18:1/18:0/16:0), TAG 54:6 (20:4/16:0/18:2), TAG 54:5 (18:2/18:1/18:2), TAG 54:4 (18:1/18:2/18:1), TAG 54:3 (18:2/18:0/18:1), TAG 54:2 (18:1/18:0/18:1), TAG 54:1 (18:0/18:1/18:0), TAG 56:8, TAG 56:7 (20:1/18:1/18:2), TAG 56:5 (18:1/20:3/18:1), TAG 56:4 (18:2/20:2/18:2), TAG 56:2 (18:1/20:0/18:1), CE 16:1, CE 16:0, CE 18:3, CE 18:2, CE 18:1, CE 18:0, CE 20:5, CE 20:3, CE 20:2, CE 20:1, CE 22:6, CE 22:5, and CE 22:4 is increased compared to the control.

10. The method of determining the severity of pre-eclampsia of claim 9, further comprising determining that the subject is diagnosed as having severe pre-eclampsia, in case that the levels of the one or more lipidomes and LPC 16:0 are increased compared to the control.

11. The method of determining the severity of pre-eclampsia of claim 5, wherein the lipidome is one or more selected from the group consisting of LPC 20:4, LPC 20:3, LPE 20:4, LPE 22:6, PE 40:5, Plasmenyl PC 36:5, Ceramide 36:1 (d18:1/18:0), SM 32:1, SM 36:1, SM 38:4, SM 38:1, DAG 32:0 (16:0/16:0), DAG 36:1 (18:0/18:1), TAG 46:1 (14:0/16:1/16:0), and TAG 46:1 (14:0/18:1/14:0).

12. The method of determining the severity of pre-eclampsia of claim 11, further comprising determining that the subject is diagnosed as having non-severe pre-eclampsia:
in case that the level of one or more lipidomes selected from the group consisting of LPC 20:4, LPC 20:3, LPE 20:4, LPE 22:6, PE 40:5, Ceramide 36:1 (d18:1/18:0), SM 36:1, SM 38:1, DAG 32:0 (16:0/16:0), and DAG 36:1 (18:0/18:1) is increased compared to the control; or
in case that the level of one or more lipidomes selected from the group consisting of Plasmenyl PC 36:5, SM 32:1, SM 38:4, TAG 46:1 (14:0/16:1/16:0), and TAG 46:1 (14:0/18:1/14:0) is decreased compared to the control.

13. A kit comprising an agent for measuring a level of a lipidome, wherein the lipidome is one or more selected from the group consisting of CE 16:1, CE 16:0, CE 18:1, CE 18:0, CE 22:5, CE 22:4 and DAG 36:1 (18:0/18:1).

14. The kit of claim 13, wherein the kit for diagnosing pre-eclampsia or determining the severity of pre-eclampsia.
